(19) United States
(12) Patent Application Publication
(10) Pub. No.: US 2008/0161396 A1
(43) Pub. Date: Jul. 3, 2008

(54) Method of control of arthropod pests of game and pet birds

(76) Inventors: J. Allen Miller, Kerrville, TX (US); J. Mathews Pound, Ingram, TX (US)

Correspondence Address: USDA-ARS-OFFICE OF TECHNOLOGY TRANSFER;NATIONAL CTR FOR AGRICULTURAL UTILIZATION RESEARCH, 1815 N. UNIVERSITY STREET, PEORIA, IL, 61604, US

(21) Appl. No.: 12/001,802

(22) Filed: Dec. 13, 2007

Related U.S. Application Data
(60) Provisional application No. 60/875,928, filed on Dec. 20, 2006.

(51) Int. Cl. A61K 31/216; A61P 33/00

(52) U.S. Cl. 514/531

(57) ABSTRACT

Ticks and other arthropod ectoparasitic pests of birds may be effectively controlled and the populations of ticks on treated birds significantly reduced by affixing to the bird a leg band or wing tag containing a pesticide in an amount effective against the tick or other pest.

### CROSS-REFERENCE TO RELATED APPLICATION

This application claims the benefit under 35 U.S.C. 1.19(e) of U.S. provisional No. 60/875,928, filed Dec. 20, 2006, the contents of which are incorporated by reference herein.

### BACKGROUND OF THE INVENTION

1. Field of the Invention

The invention is drawn to a method of controlling arthropod ectoparasites on birds.

2. Description of the Prior Art

Disease transmission between domestic animals and wildlife can be a source of human-wildlife conflict either when transmission occurs from a wildlife host to livestock or from domestic animals to endangered wildlife (Cleveland et al., 2001). Ixodid ticks are important arthropod vectors of diseases that cause significant mortality, morbidity and economic loss to human, livestock and wildlife hosts in the tropics and temperate parts of the world (Sonenshine & Mather, 1994) with annual costs of losses and control amounting to billions of dollars (Sonenshine, 1991). The sheep tick (Ixodes ricinus) is the only species of major economic and pathogenic importance in Great Britain, being the principal vector of pathogens causing several diseases including Lyme disease and louping ill (Sonenshine, 1993).

Of particular interest in the Scottish uplands is the role that the sheep tick may have in the decline of red grouse (Lagopus lagopus scoticus). Red grouse in the UK are characterized by a regular cycles in population size accompanied by long-term decline in overall populations over recent decades (Shaw, 2004). The factors influencing the density of red grouse populations are varied, complex and only partially understood (Hudson, 1992; Hudson et al., 1998; Thirgood et al., 2000; Moss & Watson, 2001; Smith et al., 2001) but it is thought to be due to habitat loss through land use change (Krebs & May, 1990) although other factors such as predation (Redpath, 1991) and disease (Hudson, 1992) have been locally important. Management of red grouse aims to maximize populations by providing conditions that reduce predation and parasitism, and optimize food quality and habitat structure suitable for breeding and recruitment. On Moors where best practice management techniques are implemented the failure of grouse populations to respond is often attributed by managers to poor recruitment due to high chick mortality because of tick infestation and the associated transmission of louping ill virus (LIV).

Typically, ixodid ticks follow a three-host life-cycle, with the larva, nymph and adult taking a blood meal from a different type of vertebrate host at each developmental stage. The blood meal of an adult female tick, for egg production, is taken from a large mammalian host. In the Scottish uplands, these hosts are typically red deer (Cervus elaphus L), roe deer (Capreolus capreolus L.) or sheep (Ovis aries L) (Wilson et al., 1988; Gray et al., 1992; Kurtenbach et al., 1995; Hudson et al., 2001). Although low tick burdens do not have any clear impact on host growth and survival (Hudson 1986, 1992), tick transmitted LIV can cause significant losses in sheep and red grouse (Reid, 1975; Reid et al., 1978) and could be a limiting factor for grouse populations (Laurenson et al., 2003). Mortality rates in grouse infected with LIV are 79% (Buxton and Reid 1975; Hudson et al., 1997; Gilbert et al., 2001) with the earliest clinical signs occurring five days post-infection (Buxton and Reid 1975). Infection with LIV may also negatively affect chick weight compared to non-infected chicks (Reid et al., 1978). Even in the absence of louping ill, tick infestations can have a debilitating effect on chicks (Duncan et al., 1978; Kirby, 2003 [thesis]). Increased tick parasitism weakens chicks through anaemia, reduces feeding through eye closures, and facilitates increased tick feeding as immunity is suppressed.

The increasing importance of ticks is supported by a recent study which demonstrated that tick infestations of red grouse chicks (aged 1-40 days) has increased between 1985 and 2003 from an average of 2.6 to 12.71. The number of parasitized chicks caught also rose during the same period (Kirby et al., 2005). There are at least two causes of this increase. First, populations of the definitive tick hosts such as deer have been increasing in the Scottish uplands (Fuller & Gough, 1999; Clutton-Brock et al., 2004). Changes in deer density could create conditions that increase the distribution of and prevalence of tick borne diseases with consequences for humans and wildlife. This could be a reason for the declining grouse populations in recent years. Second, recent rises in temperature in the Scottish uplands (SENS, 2002) is likely to benefit tick populations by extending the tick questing season and increasing over-winter survival as well as creating suitable conditions for new foci (Brownstein et al., 2003).

To date, tick control strategies have focused on sheep or reductions in wild definitive tick hosts. Good sheep management through vaccination and tick control is still fundamental to the control of louping-ill virus and its tick vector. In many cases sheep flocks are managed to reduce tick abundance on the Moor (tick mop flocks) by regularly treating them with acaracide, which kill questing ticks on contact. It has been demonstrated that this will work if strictly managed over a period of years when no other tick hosts are present (Moors in the North of England) but its effectiveness when coexisting hosts (deer and hares) are present may take much longer.

Despite costly vaccination and acaricide treatments of adult sheep for over 30 years, the disease is poorly controlled in red grouse in certain regions of the Scottish Highlands (Hudson et al., 1995). Experimental reductions of hares in the absence of sheep and deer have led to declines in tick abundance and louping ill with corresponding increases in grouse populations (Laurenson et al., 2003). However, modeling suggested that hare culls are unlikely to be effective in other areas where deer are present even at low densities has been questioned. “Where grouse and red deer were the only hosts several combinations of the virus could lead to the virus persisting or dying out. At low deer densities, the virus could not persist since there were then too few hosts for adult ticks to maintain the tick population. At extremely high densities, the virus tended to die out due to a ‘dilution’ effect. However at more realistic deer densities the disease was likely to persist due to the combined effect of deer amplifying the tick population and grouse transmitting virus” (Gilbert et al., 2001). Even where tick numbers are in decline, there is even less evidence that grouse production will necessarily increase, presumably because other factors affecting recruitment may be more important. Thus, in some cases, reduced grouse production has prompted expensive tick control management strategies or the culling of alternative hosts when it is not clear that ticks are the cause of the poor chick recruitment.

To date there have been no studies that have been able to determine the relative importance of the different factors affecting the recruitment of chicks to the breeding population. What is needed is to investigate the causes and timing of chick losses and determine the extent to which ticks are responsible for these losses. Chick losses can be due to a number of factors including hen condition (affected by nematode burdens and food quality), egg quality and hatching success (affected by hen condition and quality), and post hatching chick survival (affected by weather, invertebrate food abundance, tick worry and louping-ill). While there is some evidence that treating hens and chicks with acaracide can reduce tick infestation rates and increase short-term survival, the results were inconclusive due to small sample sizes and problems with chick mortality caused by the acaracide application method (Laurenson et al., 1997). Thus, the need remains for improved methods of controlling ticks and other arthropod pests on birds.

### SUMMARY OF THE INVENTION

We have now discovered that ticks and other arthropod ectoparasitic pests of birds may be effectively controlled and the populations of ticks on treated birds significantly reduced by affixing to the bird a leg band or wing tag containing a pesticide in an amount effective against the tick or other pest. The method is particularly effective for the control ectoparasites on game and pet birds, and most particularly for the control of ticks on adult female nesting broods and their chicks.

In accordance with this discovery, it is an object of this invention to provide a method for controlling arthropod pests on birds.

Another object of this invention is to provide a method for controlling ticks and other arthropod ectoparasites in game and pet birds.

A further object of this invention is to provide a method for controlling ticks and flies which exhibit significantly greater efficacy than those utilizing organophosphate pesticides alone.

Yet another object of this invention is to provide methods and compositions for controlling ticks and other arthropod ectoparasites on adult female nesting birds and the chicks in their nests.

Other objects and advantages of this invention will become readily apparent from the ensuing description.

### BRIEF DESCRIPTION OF THE DRAWINGS

FIG. 1 shows the seasonal pattern of tick infestation of red grouse chicks for the three treatment groups. Thick lines denote the predicted values and thin lines in the same group represent the confidence limits.

FIG. 2 shows the relationship between tick infestation rates and chick age for the three treatment groups. Thick lines denote the predicted values and thin lines in the same group represent the confidence limits.

FIG. 3 shows the mean brood size at difference stages for both control and treated hens (data for both moors combined).

FIG. 4 shows the growth curves of (a) wing and (b) weight of chick from control (open symbols) and treated broods (filled symbols). Data from Moor 1 & 2 combined.

FIG. 5 shows the growth curves of chicks (body mass) from Moor 1 and 2 in their first 10 days compared with the growth from chicks reared in captivity under three diet type: 1) heather and unlimited invertebrate (solid line); 2) heather and limited invertebrate (long dashed line); or 3) heather and no invertebrates (short dashed line; those chicks did not survive more than a week). Data on chick growth from captive chicks is from Park et al. (2001).

FIG. 6 shows the survival (% alive) of treated and control females on each study Moor.

FIG. 7 shows the rainfall summed over 10 day periods plotted with a) mean hatching success and b) mean brood survival to two weeks for the same periods.

### DETAILED DESCRIPTION OF THE INVENTION

Application of pesticide containing wing tags or leg bands of the invention is effective for controlling ticks and other arthropod ectoparasites on a variety of birds, but is preferably used for the treatment of game and pet birds, including but not limited to quail, pheasants, grouse, canaries, parakeets, macaws, and parrots, and most preferably the Red Grouse. Moreover, while the method may be used for the treatment and control of arthropods on male or female birds at virtually any age from chicks to young birds to adults, we have found that the method is particularly suited for controlling arthropods on adult female nesting birds (i.e., mother hens) and their chicks. For the birds to which the pesticide-containing leg bands have been attached, as the birds nest with their legs beneath their plumage or use their legs to scratch, the pesticide is distributed into and over the plumage. Similarly, for the birds carrying the pesticide-containing wing tags, the pesticide is distributed by movement of the wing against the plumage or the head coming in contact with the wing. However, we have also discovered that when the wing tags, and preferably the leg bands, are affixed to adult female nesting birds, as the treated mother sits on her nest or gathers her chicks, the pesticide is applied to the nest and the chicks, thereby serving to reduce tick and other arthropod populations and the concomitant transmission of ectoparasite-carried diseases to the chicks as well.

The method effectively controls a variety of arthropod pests on the treated birds, including insect and acarine ectoparasites, and preferably ticks, mites, and lice. In a particularly preferred embodiment, the method is used for controlling ticks on the Red Grouse, particularly ixodid ticks such as the sheep tick (Ixodes ricinus) thereon which are vectors of louping ill disease in these birds in Scotland.

The timing of the application, including the age of the birds and the season of the year of the pesticide-containing leg bands and wing tags to the birds, will vary with the pest, the geographic area, and the pest's native cycle, and may be readily determined by the practitioner skilled in the art. For example, without being limited thereto, for Red Grouse in Scotland, the tags or bands are preferably applied in the spring and remain on the birds until late summer/early fall. In contrast, for pet birds such as parakeets, parrots, and macaws, the tags or bands may be applied at any time of the year to protect against ticks, lice, and mites.

A variety of pesticides are known for control of arthropod ectoparasites such as insect and acarine pests, and particularly ticks, lice, and mites, and are suitable for use with the wing bands or ear tags herein, and include insecticides, acaracides, and miticides. The optimum pesticide selected will vary with the particular target arthropod pest. Without being limited thereto, suitable pesticides include organophosphates such as coumaphos or diazinon; pyrethroids such as permethrin, cyfluthrin or cypermethrin; avermectins such as ivermectin, doramectin or eprinomectin; formamidines such as amitraz; and combinations thereof. Permethrin is preferred.

The material or materials of construction of the wing tags and leg bands are not critical, although at least a portion of the tags or bands should be capable of being impregnated, laminated or coated with a suitable, conventional pesticide. The tag or band may be constructed from a variety of different materials, including those conventional in the art, and they may be flexible or inflexible. Suitable materials include, but are not limited to metals, synthetic or natural polymers such as rubbers, plastics or foams, particularly polyvinylchloride or ethylene vinyl acetate, synthetic or natural fabrics such as nylon or cotton (including cotton cord), felt or wool, leather, or combinations of the above. However, soft materials minimize chafing about the animal's legs and wing and are generally preferred. The shapes of the leg band or wing tag are not critical and they may have a round or flat cross section, although flat cross sections are generally preferred as they provide a greater surface area for contact with the body of the subject bird, nest, and in the case of adult female nesting birds, the hatched chicks in the nest as the mother hen roosts over them. The tags and bands may be constructed as a single, one piece unit, or it may be constructed from multiple pieces joined together. Tags and bands may be secured to the bird's wing or leg also using techniques conventional in the art, such as by conforming it around the leg or by use of adhesives, anchor straps, ties, zip closures (such as found on common cable zip-ties), punches, cooperating hook and loop type fasteners (such as VELCRO), clips, snaps or buckles.

The pesticide carried by the wing tag or leg band may be in a liquid or dry formulation. As a practical matter, it is anticipated that the pesticide will be prepared by formulating with a suitable inert carrier as known in the art. The pesticides may, for example, be formulated as solutions, emulsions, emulsifiable concentrates, suspension concentrates, wettable powders, dusts, granules or adherent dusts or granules. Of greatest interest are those carriers which are agronomically or pharmaceutically acceptable, particularly those suitable for topical application onto animals. The particular carrier selected is not critical, and a variety of liquid and solid phase carriers may be used, including but not limited to water, aqueous surfactant mixtures, alcohols, ethers, oils, hydrocarbons, halogenated hydrocarbons, glycols, aldehydes, ketones, esters, oils (natural or synthetic), clays, kaolinite, silicas, cellulose, rubber, talc, vermiculate, and synthetic polymers. However, oils and volatile solvents such as acetone or isopropyl alcohol are generally preferred. Alternatively, the pesticide may be formulated directly into molten polymers. This is particularly preferred when the molten polymers are to be subsequently extruded to form the wing tag or leg band. It is also understood that the pesticide may also be formulated into conventional controlled release microparticles or microcapsules.

The leg band or wing tag may be impregnated or coated with either a liquid or dry formulation of the pesticide. In accordance with one preferred embodiment, the tag or band may include an absorbent material which is saturated with a liquid composition of the pesticide. In this embodiment, the user may optionally saturate the absorbent material with fresh liquid at periodic intervals. As mentioned, in an alternative preferred embodiment, the pesticide may be mixed into a molten polymer melt which may then be extruded to form the wing tag or leg band. Alternatively, liquid or dry formulations may be coated directly onto the surface of the wing tag or leg band. In another alternative embodiment, the leg band or wing band may include plastic strips impregnated with the chemical agent which are attached thereto or wrapped around an inner core. For pesticide application, suitable applicators for use herein include but are not limited to Taktic strips (Hoechst Roussel Agri Vet Co., Sommerville, N.J.) impregnated with amitraz.

Radio transmitters and/or identification indicia may also be included on the wing tags or leg bands for tracking or managing bird populations.

The pesticide in the wing tags or leg bands of the invention generally act to control pests by killing the targeted tick, mite or other insect or acarine pest. Accordingly, the pesticide on or in the tag or band is provided in an amount effective to induce death of the targeted tick, louse, mite or pest as predetermined by routine testing. An “effective amount” or “pesticidally effective amount” is defined herein to mean those quantities which will significantly reduce or eliminate the population of the target arthropod (e.g., tick) on birds in a treated or test group as compared to an untreated control group (measured at a confidence level of at least 80%, preferably measured at a confidence level of 95%). Suitable amounts and concentrations may be readily determined by a practitioner skilled in the art, and will vary with the particular species of pest and its strain (i.e., resistant or relatively sensitive to the selected pesticide), its stage of development, the particular pesticide, the type of vehicle or carrier, and the desired duration of treatment. By way of illustration and without being limited thereto, preferred compositions of the invention include permethrin formulated in a PVC matrix in a cord form approximately 3 mm in diameter and 13 cm in length for attachment to the legs of Red Grouse or in tape form approximately 1 mm in thickness and 3 cm in length for the wing tag.

The following example is intended only to further illustrate the invention and is not intended to limit the scope of the invention which is defined by the claims.

### EXAMPLE 1

This experiment assessed the effect of ticks on grouse productivity. The experiment was replicated in two sites located in NE Scotland and Perthshire. On each Moor, hens were either treated with permethrin leg bands or with untreated bands. After hatching, chicks from treated hens were also treated and tagged with a permethrin band attached to a metal patagial tag, while chicks from control broods were tagged with only the metal patagial tags.

### Materials & Methods

The experiment was conducted on two sites located in the North of Scotland (March-October). In outline, on each Moor, hens were either treated with permethrin leg bands or with untreated band. After hatching, chicks from treated hens were also tagged and treated with permethrin partagial bands, while chicks from control brood were tagged but not treated with permethrin. The experiment was conducted on Tullybeagles Estate in Perthshire (hereafter referred as to Moor 1) and Forest of Birse Estate on Deeside (hereafter Moor 2). Both Moors are managed for grouse (heather burning, predator control, worm control) and had low grouse densities and low productivity in recent years.

Catching and treatment of hens. In spring (18 March-6 April), 40 hens were caught on Moor 1 and 20 on Moor 2. Each was randomly assigned to one of two treatments. Half the birds caught at each site were treated (i.e., given acaracide leg bands, two per bird), or kept as control (untreated bands). Hens were fitted with radio collars (TW-3, Biotrack) to facilitate relocation. Birds were aged, weighed, wing length measured and condition score recorded. All hens were dosed with anthelminthic (Levamisole hydrochloride 3%) to remove nematode parasites. Nematodes are well known for their negative effects on grouse breeding success and survival (Hudson, 1986), so removing them standardized the birds for this possible source of variation.

Laying time. Nests were located by radio tracking and their location recorded using GPS. Clutch size was measured, and all eggs were weighed (with an electronic balance, to the nearest 0.1 g) and measured (length by width, with a caliper, to the nearest 0.1 mm). Egg density was used to estimate hatch dates (see Seivwright, 2004). If clutches were incomplete at first visit, nests were revisited.

Hatching success. Around predicted hatch date (from egg measurements), nests were located using GPS and number of hatched and un-hatched eggs were recorded. Broods were located by radio-tracking hens and chicks were found and counted with the aid of trained pointer dogs.

Brood survival to 2 weeks +/−5 days and treatment of chicks. Hens were located by radio tracking and pointer dogs were used to locate chicks. As many chicks as possible were captured. Body mass, wing Brood survival to 1 month days +/−5 days. Hens were located by radio tracking and pointer dogs were used to locate chicks. As many chicks as possible were captured. Body mass, wing length and tick counts (around head and neck area) were recorded. A blood sample was collected in EDTA coated eppendorfs. At 1 month, because it was difficult to ensure that all the chicks were found during daytime visits with dogs, we also located hens and broods at night by radio tracking and broods were counted using lamp. This provided the best estimate of brood size at 1 month, which was used in subsequent analyses.

Brood size at fledging. Hens were located at around 45 days post hatching. Broods were then flushed and number of chicks was counted.

Blood samples and LIV prevalence tests. Blood samples were collected from the brachial vein from chicks at each catching occasion and from hens in autumn (when the radios where retrieved). Chicks less than 2 weeks of age were not all sampled in order to minimize unnecessary trauma in small chicks. Samples were centrifuged and the plasma was separated within 6 hours of collection. Samples were then frozen and transported to Moredun Institute and kept at −70° C. Plasma was used into determine seroprevalence for louping-ill virus using standard methods (Reid et al., 1978)

Weather data. Rainfall data for Bankfoot Automatic weather Station was downloaded courtesy of the British Atmospheric Data Centre. The hatching period was divided into 10 day periods and the sum of rainfall for those periods was calculated. The mean hatching success and survival to 2 weeks was calculated for the same periods.

Statistical analyses. Tick numbers were modeled using generalized linear mixed models with brood as a random effect and using a poisson error distribution. Tick burdens are known to be much less variable within broods than between broods (Elston et al., 2001). The seasonal pattern was investigated by fitting time as julian day (days from 1^{st} January) and the asymptotic pattern in the data was captured using a quadratic function of julian day (julian day*julian day). The interaction between time and treatment was used to determine if the pattern of tick burden varied between treatments. The relationship between tick burden and age was investigated using chick age as number of days post hatching and the quadratic function of chick age to investigate if there was a significant asymptotic relationship between average brood tick abundance and chick age (a rise and fall in tick numbers in relation to age). Hatching success was modeled using logistic regression (binomial error structure) with the proportion of eggs that hatched as a proportion of the total clutch size as the response variable. Brood survival was modeled using logistic regression (binomial error structure) with the proportion of chicks alive as a proportion of the number of chicks that hatched as the response variable.

Caveat on brood size. Chick counts at any one time are likely to under estimate the number of chicks alive. It is therefore possible for later brood counts to be greater than earlier ones. Thus earlier counts were revised upwards if later counts revealed more chicks than were previously detected.

Caveat on chick acaracide treatment. Because not all chicks could be captured at the first visit, not all the chicks from a treated hen were treated. Thus it is difficult to determine individual chick survival particularly when broods were only flushed and not handled. However, untreated chicks from treated hens allowed us to test the effect of hen treatment alone on tick infestation rates.

### Results

### Effect of Treatment on Tick Infestation Rates

a) The Seasonal Pattern in Tick Abundance (Tick Rise).

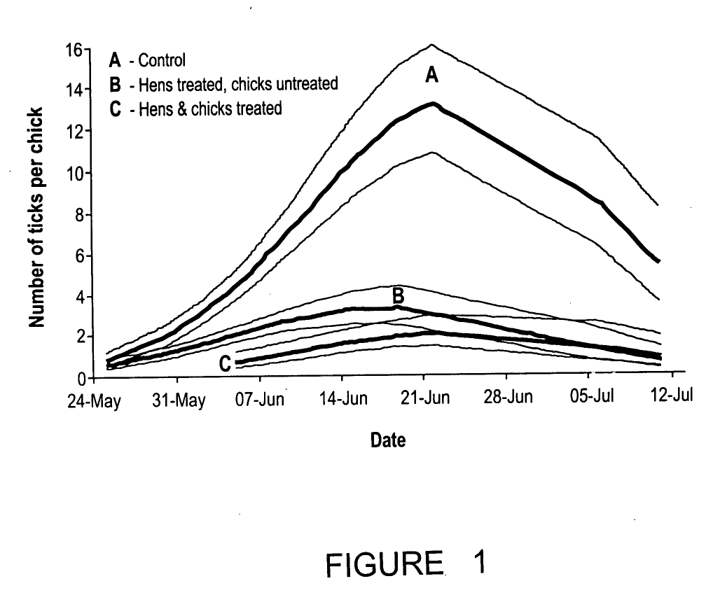

Prior to testing for treatment effects, we looked at seasonal variation in tick burdens on chicks, using the broods from control hens only (FIG. 1, black line). Tick numbers were very low in the first chicks caught in late May. They peaked in mid June with a mean of around 13 ticks per chick and declined markedly by mid July. The most infected chicks had up to 57 ticks on Moor 1 and 45 ticks on Moor 2. Thus, chicks born in late May are unlikely to be exposed to high numbers of questing ticks. However, chicks born late (in early to mid June) will potentially be exposed to high tick abundance. There was no difference between Moors in relation to the pattern of tick infestation over time (F1, 51.4=1.55, p>0.21).

b) Relationship Between Tick Infestation Rate and Chick Age.

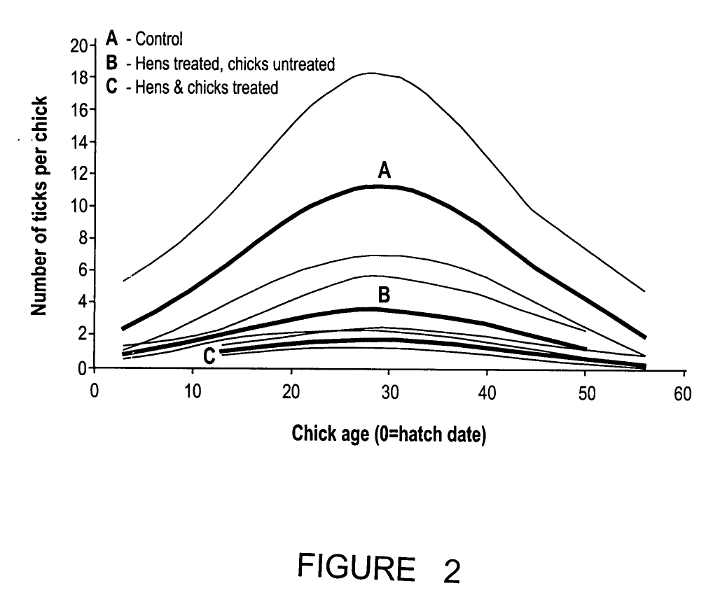

Again using control broods (FIG. 2, black line), it is possible to determine the pattern on tick infestation in relation to chick age. From this, it is clear that young chicks all have low tick burdens despite some of them having hatched as late as mid June. Chicks caught later show increasing tick infestation rates which peak at about 4 weeks of age with a mean of around 11 ticks per chick. There was no difference between Moors in relation to the pattern of tick infestation as chicks aged (F1, 58.2=0.26, p>0.61).

c) The Effect of Acaracide Treatment of Chicks on Average Brood Tick Infestation Rates.

Taking into account the seasonal pattern in tick abundance, average tick counts on treated chicks were significantly lower than control chicks (F1, 91.9=44.27, p<0.01, FIG. 1, compare blue and black lines). Average tick numbers per chick in the treated group reached a peak of only 2 ticks per chick per brood (FIG. 1, blue line). In fact, only one chick that had a patagial band treatment band was found with ticks, and ticks were sometimes found on chicks that had not yet been treated or that had lost their patagial band. The same result was true when taking into account the pattern of tick abundance in relation to age. Treated chicks had significantly lower tick numbers than controls (F1, 117=32.47, p<0.01 FIG. 2, compare blue and black lines) throughout the age range (0 to 55 days of age) with maximum mean infestation rates in the treated group less than 2 ticks per chick (FIG. 2, blue line) compared to around 12 for control broods (black line).

d) The Effect of Acaracide Treatment of the Hen on Tick Abundance in Young Chicks.

Average ticks per chick in untreated chicks from treated hens were significantly lower then tick abundance on control broods (F1, 68=19.8, p<0.01, FIG. 1 red versus black line) across the season. Tick infestation rates between treated chicks were significantly lower than those found in untreated chicks from treated mothers (F1, 275=7.99, p>0.01, FIG. 1, blue versus red line). Peak tick numbers in the untreated chicks from treated hens were around 4 ticks per chick about one third of that found in controls. The site by time interaction was not significant indicating that the pattern of tick abundance did not differ between Moors in relation to season. The same pattern was observed for tick numbers in relation to chick age. Tick infestation in untreated chicks from treated hens was significantly lower than those found in controls (F1, 117=17.57, p>0.14, FIG. 2, compare blue and black lines) but there was no significant difference in tick infestation rates between treated chicks and untreated chicks from treated mothers (F1, 117=2.1, p>0.14, FIG. 1, blue versus red line). Thus treating hens provides protection from tick infestation that was similar to that found in treated chicks when compared age for age (FIG. 2, age) but did not provide quite as good protection when comparing over the same time period (FIG. 1, season). The site by chick age interaction was not significant indicating that the pattern of tick abundance did not differ between sites in relation to chick age.

Effect of treatment on breeding success, chick growth and survival

Prior to testing for treatment effects on breeding success, we compared breeding inputs. Laying date and clutch size did not differ between treatment groups or Moor (Table 1).

a) The Effect of Hen Treatment on Hatching Success

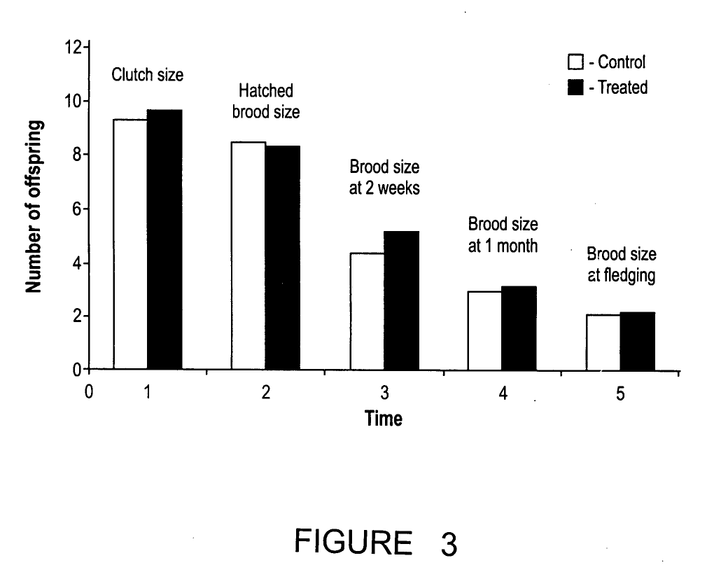

There was no effect of hen treatment on the number of eggs that hatched as a proportion of the clutch size. Clutch size for controls and treated hens was 9 and 9.8, respectively, at Moor 1 and 9.5 and 9.45 for Moor 2 and 10.2 (See Table 1). There was no significant effect of treatment or site on hatching success (Table 1; FIGS. 3a & b).

### b) The Effect of Treatment on Chick Survival

Brood size at 2 weeks of age. There was no effect of treatment on brood size at 2 weeks of age (Table 1; FIG. 3). However, there was a site effect indicating that brood size was significantly lower at Moor 2 than in Moor 1 (Table 1). The interaction between site and treatment was not significant which indicates that there was no evidence for a treatment effect at either site after taking into account site differences.

Brood size at 1 month of age. There was no effect of treatment on brood size at 1 month of age (Table 1, FIG. 3). There was still a tendency for bigger broods on Moor 1 than on Moor 2 (Table 1). The interaction between site and treatment was not significant which indicates that there was no evidence for a treatment effect at either site after taking into account site differences.

Brood size at fledging. Broods were counted at time of year that grouse productivity is normally measured (late July). There was no effect of treatment or difference between sites in brood size at fledging (Table 1, FIG. 3).

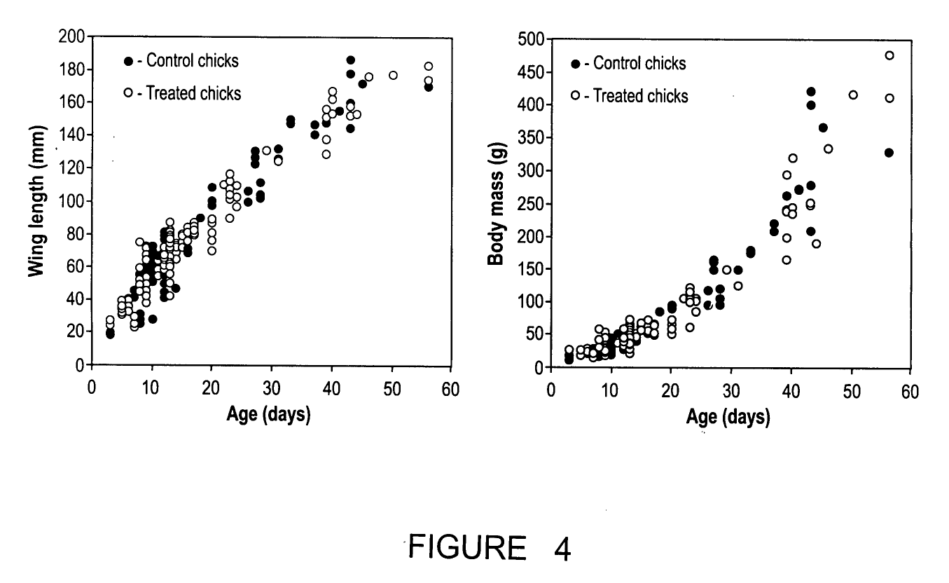

Chick growth. We found no effect of treatment on the growth (wing length or body mass) of chicks (data shown in FIG. 4).

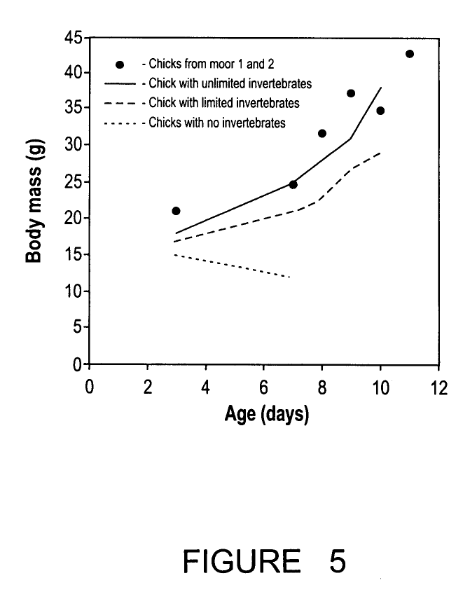

In order to evaluate whether food limitation, and the lack of invertebrates in particular, might have been a problem for chick growth, we compared the growth data from Moors 1 & 2 with those from experiments done under controlled food conditions on captive reared chicks (data in Park et al., 2001). Growth rate (body mass) did not differ between Moors 1 and 2, and did not differ between control and treated broods, in either Moor. Thus, average body mass was used at a given chick age using all the data from Moor 1 and 2, and from control and treated broods. The comparison with the data from Park et al., 2001 (FIG. 5) showed that the growth of chicks was not different from that of chicks reared in captive conditions with unlimited invertebrate in their diet. This suggests that food limitation, and the lack of invertebrates, was not occurring and that all chicks did show typical normal growth curves on both Moor 1 and Moor 2.

Timing of chick losses. The initial loss of chicks comes from the failure of hens to reproduce. This occurred in 9 out of 40 hens on Moor 1 (8 predation and one hen that failed to lay) and 8 out of 20 at Moor 2. On Moor 2, 6 mortality cases occurred before or soon after hatching, one occurred 15 to 30 days post hatch, one occurred 30+ days post hatch, so most cases of predation probably resulted in the loss of broods. Hatching success was very high at greater than 95% and there was no difference between sites. The greatest losses of hatched chicks occurred between hatching and approximately 2 weeks of age when the chicks were first located. Only 58% (CL=49-66%) of the hatched chicks were alive in the control group and 65% (CL=57-71%) in the treated group. Chick losses continued thereafter and at fledging less than 30% of the hatched chicks were still alive at Moor 1.

c) LIV Seroprevalence in Chicks.

The prevalence of LIV was zero in tested chicks at Moor 2 and 1.4% (1/71) at Moor 1. Therefore, at both these sites, LIV in grouse chicks is very low or non-existent. It is not possible to test the impact of acaracide treatment on LIV prevalence at these two sites.

### Hen Survival

a) Effect of Treatment on Hen Survival

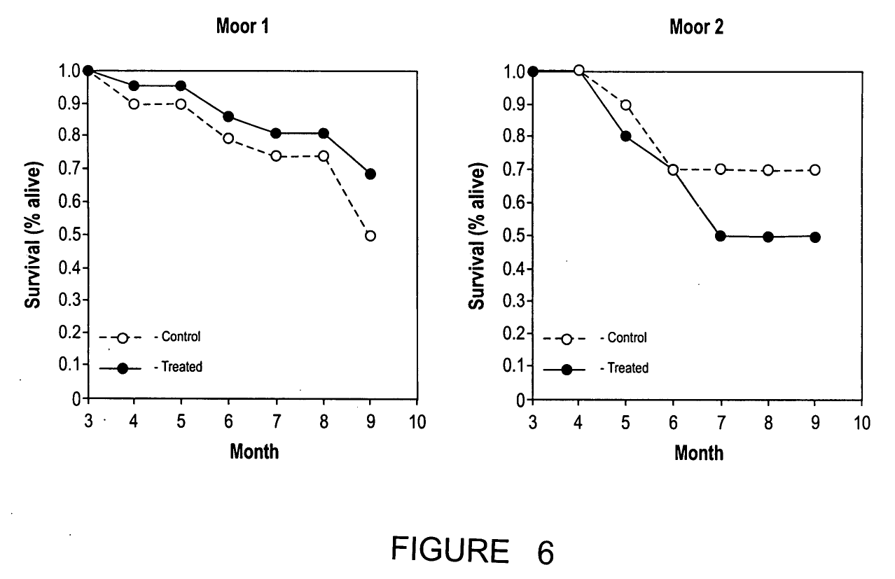

Overall, survival did not differ between treated and control hens (FIG. 6). On Moor 1, treated hens tended to survive better than control hens (50.0% and 68.4% of control and treated hens were alive in October, respectively), but the reversed trend was observed on Moor 2 (70% and 60% of control and treated hens were alive in October, respectively).

b) Causes of Mortality

The cause of death could be assessed for 18 out of 21 hens found dead. Identified causes are listed in Table 2. Predation appeared to be the commonest cause of death (13 out of 21 cases; 61.9%). One possible case of death by LIV was detected on Moor 2.

### Discussion

Effectiveness of the treatment. Directly treating chicks with acaracide reduces tick infestation to negligible levels. Tick infestation levels in untreated chicks from treated hens were also very low although significantly higher than those found in treated chicks. Control broods had much higher tick infestations reaching a mean peak of around 11-13 ticks per chick per brood. Although acaracide treatment was successful in reducing tick infestation rates to very low levels, there was no effect of treatment on chick survival rates. The brood sizes at all stages from hatching to fledging were similar in both controls and treated hens (Table 1). Greatest losses of chicks from hens that bred occurred between hatching and 10 days of age (around 40% and 70% of the potential annual production, Moor 1 and Moor 2 respectively). The results from this study demonstrate high levels of chick mortality and that in the group where tick infestations were reduced, there was no significant improvement in survival.

Hen losses. A major loss of potential recruitment is hen mortality before and after hatching, or when chicks are still young. At Moor 1, eight out of 40 hens died and one hen failed to lay a clutch. This represents about 20% loss of potential annual production and this was even greater at Moor 2 where 40% of the potential production was lost due to hen mortality. This means that the remaining hens need to produce at least 2.5 chicks on Moor 1 for the population to remain stable (excluding the effect of over-winter mortality and hunting). Estimates from this experiment indicate that brood sizes at fledging are between 2.3 and 2.7 (+/−0.75) at Moor 1 which is barely enough for the population to remain stable. At Moor 2, the brood sizes are even lower which has implications for population recovery at this site. Raptors contributed to some of the losses, but mammal predation also had a significant impact (mainly by stoat, and less often by foxes).

Chick losses. Some studies have suggested that variation in chick survival between sites influences productivity, and other studies suggest that chick survival within a site may play a role in year-to-year changes in grouse numbers. (Jenkins, Watson & Miller, 1963, 1967; Watson & Moss, 1979; Moss & Watson, 1985; Hudson, 1986a; Hudson, 1992, Hudson et al., 1997, Watson et al., 1994). The first 2 weeks of life is the time when the main mortality of chicks occurs even on Moors with no ticks (Jenkins, 1963) and can be due to a number of causes including weather, diet quality, predation, ticks and LIV.

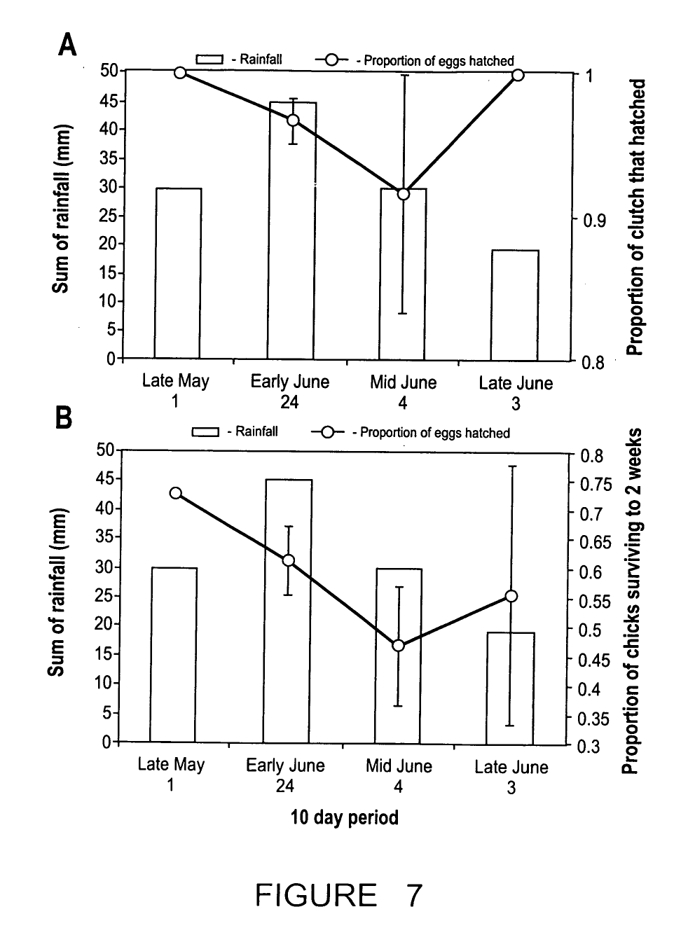

Chick losses—Weather. This study confirms earlier studies in demonstrating that the bulk of chick losses occur in the first 2 weeks of life. Losses may be due to extrinsic effects such as such as rainstorms during hatching. Bad weather around the time of hatching may explain the heavy losses observed in the first 2 weeks of chicks on both Moors. For example, during one rain storm one nest was located on the hatch date and had been abandoned by the hen. Water was running through it and five of the chicks had hatched but were lying dead by the nest. The remaining 4 eggs were cold and unhatched. FIG. 7 illustrates that there is no obvious association of poor survival with rainfall. However, this is difficult to quantify or demonstrate. More than one year of data would be required to better evaluate this cause of chick loss.

Chick losses—Ticks. Chick losses during the first 2 weeks ranged from 40% on Moor 1 to 70% on Moor 2. Although we were unable to locate dead chicks, chick losses during the first 2 weeks are unlikely to be due to ticks because tick infestation rates were low early in the season and in young chicks whenever they are born (FIGS. 1 & 2). Unpublished data from Lochindorb Estate confirms this pattern (L. Gilbert pers comm.). Furthermore, the analysis demonstrated that at no stage were brood sizes higher in treated animals than controls despite large differences in tick infestation rates between treated and control birds. Tick infestation rates peaked at around a mean of 11-13 per chick in the control chicks. Compared to previous years for this site, tick burdens were relatively lower than in previous years, although it compares well with the ticks per chick in 2003 reported in Kirby (2004; i.e., averages of 12-13 ticks per chicks). Although a previous study showed that there were short term effects lasting up to 20 days using a pour on deltamethrin on a high tick site, the effect had waned by 40 days (Laurenson et al., 1997). It is well established that the number of ticks per chick was negatively correlated to the number of young reared per hen and the breeding density of adult grouse and that there was a stronger relationship for nymphs than larvae. In another study, 14 chicks less than 2 weeks old had a mean of 90 larvae and 6.1 nymphs; their eyelids were swollen and 4 were moribund (Duncan et al., 1978). However, on both Moor 1 and 2 in the year tested, it is not clear that ticks had large deleterious effects on growth of chicks, given the lack of differences in growth rates and survival between treatment groups.

Chick losses—LIV. Louping ill can be a major cause of mortality. Earlier studies have demonstrated that up to 79% of red grouse experimentally infected with louping-ill virus in captivity died within 13 days of infection (Reid, 1975). In that study, Louping ill was a major mortality factor in the wild and the percentage of chicks positive for louping-ill virus has been positively correlated with the number of nymphs per chick. Chicks shown to have louping-ill were more likely to die than chicks where LIV was not detected (Duncan et al., 1978). The effect of treatment on LIV is difficult to determine in this study because LIV seroprevalence in grouse chicks on Moor 1 was only 1.4% (1/71) and zero on Moor 2 (although one of the hens found dead on Moor 1 was positive for the LIV). Consequently, chick losses at any stage up to fledging in this study were unlikely to be due to LIV. Losses during that crucial first 2 weeks are also unlikely to be due to LIV because any hen that has survived LIV will transmit maternal LIV antibodies to the chick thus chicks will be protected from LIV during the first 2 weeks of life. An earlier study demonstrated that little or no viral infection was found in 2 week old chicks although 2 chicks had antibodies of maternal origin (these chicks were too young to have contracted and recovered from the virus) (Duncan et al., 1978). Blood samples from hens recaptured in October will provide information on LIV seroprevalence in the experimental birds and allow us to investigate whether brood sizes at 10 days of age are larger in hens that have survived LIV. However, on Moor 1 (no data for Moor 2), data from lambs indicates that LIV has varied between 4% and 30% over the last 4 years with 26% for the year tested (Strathbraan Grouse Management Group—unpublished report).

If LIV prevalence had been higher it would have been possible to investigate whether treatment had an effect on LIV transmission. Previous studies have shown that grouse may be infected through the ingestion of infected ticks. Ticks have been found in the diet of 20% of broods and can make up 13% of the invertebrates in the diet (Hudson et al., 1997). In the lab, 50% of chicks fed with infected tick developed viraemia and infected feeding nymphs (Hudson et al., 1995). Vector ingestion may be a major route of transmission for LIV in grouse (Gilbert et al., 2004). If treated chicks had been positive for LIV, this would provide supporting evidence for the oral transmission route for LIV in the wild, but this was not observed. Reid et al. (1978) demonstrated that chicks infected with LIV weighed significantly less than uninfected ticks. Chick weights in this study compared favorably with an earlier study on captive reared, uninfected chicks fed on unlimited invertebrates (Park et al., 2001) (FIG. 5).

It is understood that the foregoing detailed description is given merely by way of illustration and that modifications and variations may be made therein without departing from the spirit and scope of the invention.

TABLE 1

| | Moor 1 | | | | Moor 2 | | | |
|---|---|---|---|---|---|---|---|---|
| | Control | | Treated | | Control | | Treated | |
| Number of hens caught | 19 | | 21 | | 10 | | 10 | |
| Number of hens that laid | 17 | | 19 | | 8 | | 8 | |
| Clutch size | 9.0 ± 1.8 | (17) | 9.8 ± 1.3 | (19) | 10.2 ± 1.3 | (6) | 9.2 ± 2.5 | (5) |
| Hatch date | 28.9 ± 5.6 | (15) | 28.2 ± 6.2 | (19) | 26.5 ± 2.2 | (8) | 27.4 ± 4.7 | (8) |
| Hatched brood size | 8.2 ± 3.1 | (17) | 8.5 ± 3.2 | (19) | 9.4 ± 1.5 | (5) | 8.1 ± 2.4 | (8) |
| Brood size at 2 weeks | 5.2 ± 2.4 | (13) | 6.0 ± 2.7 | (16) | 2.9 ± 2.1 | (7) | 3.4 ± 1.0 | (7) |
| Brood size at 1 month | 3.5 ± 2.8 | (14) | 3.6 ± 2.9 | (16) | 2.0 ± 2.5 | (7) | 2.0 ± 2.0 | (5) |
| Brood size at fledging | 2.3 ± 2.8 | (14) | 2.7 ± 2.9 | (15) | 1.9 ± 2.3 | (7) | 1.0 ± 1.2 | (5) |
| Breeding success (%)* | 37.2 ± 27.4 | (14) | 35.9 ± 27.2 | (16) | 29.5 ± 28.0 | (5) | 12.8 ± 13.7 | (5) |
| Breeding potential = Total | 153 | | 186 | | 61 | | 46 | |
| number of eggs laid | | | | | | | | |
| Breeding outcome = Total | 32 | (21.0%) | 40 | (21.5%) | 13 | (21.3%) | 5 | (10.9%) |
| number of young fledged | | | | | | | | |
| (success %) | | | | | | | | |

| | | | | |
|---|---|---|---|---|
| % hens alive after 6 months | 70% | 60% | 50% | 68% |

| | | | Moor × |
|---|---|---|---|
| | Moor | Treatment | Treatment |
| Number of hens caught | | | |
| Number of hens that laid | | | |
| Clutch size | F_{1.43} = 0.26 | F_{1.43} = 0.02 | F_{1.43} = 2.37 |
| | P = 0.61 | P = 0.88 | P = 0.13 |
| Hatch date | F_{1.46} = 0.98 | F_{1.46} = 0.00 | F_{1.46} = 0.23 |
| | P = 0.33 | P = 0.96 | P = 0.63 |
| Hatched brood size | F_{1.46} = 0.21 | F_{1.46} = 0.26 | F_{1.46} = 0.63 |
| | P = 0.65 | P = 0.61 | P = 0.43 |
| Brood size at 2 weeks | F_{1.39} = 9.24 | F_{1.39} = 0.68 | F_{1.39} = 0.01 |
| | P < 0.01 | P = 0.42 | P = 0.90 |
| Brood size at 1 month | F_{1.38} = 2.71 | F_{1.38} = 0.00 | F_{1.38} = 0.00 |
| | P = 0.11 | P = 0.97 | P = 0.97 |
| Brood size at fledging | F_{1.37} = 1.30 | F_{1.37} = 0.07 | F_{1.37} = 0.46 |
| | P = 0.26 | P = 0.80 | P = 0.50 |
| Breeding success (%)* | F_{1.34} = 1.97 | F_{1.34} = 0.68 | F_{1.34} = 0.49 |
| | P = 0.17 | P = 0.42 | P = 0.49 |
| Breeding potential = Total | | | |
| number of eggs laid | | | |
| Breeding outcome = Total | | | |
| number of young fledged | | | |
| (success %) | | | |
| % hens alive after 6 months | | | |

*Calculated using only the hens alive at the end of breeding season

TABLE 2

| | Moor 1 | Moor 2 | All |
|---|---|---|---|
| Predation by fox: | 0 (0%) | 1 (12.5%) | 1 (4.8%) |
| Predation by stoat: | 3 (23.1%) | 1 (12.5%) | 4 (19%) |
| Predation by raptors: | 3 (23.1%) | 2 (25%) | 5 (23.8%) |
| Predation (unknown | 1 (7.7%) | 2 (25%) | 3 (14.3%) |
| predator) | | | |
| Disease* | 2 (15.4%) | 1** (12.5%) | 3 (14.3%) |
| Unknown | 4 (30.8%) | 1 (12.5%) | 5 (23.8%) |
| Total | 13 | 8 | 21 |

*Bird found dead but intact
**The hen was tested positive to LIV

### REFERENCES

Baines, D., Sage, R. B. & Baines, M. M. (1994). The implications of red deer grazing to ground vegetation and invertebrate communities of Scottish native pinewoods. Journal of Applied Ecology 31, 122-131
Brownstein, J. S., Holford, T. R. & Fish, D. (2003). A climate based model predicts the spatial distribution of the Lyme disease vector Ixodes scapularis in the United States. Environmental Health Perspectives 111, 1152-1157.
Buxton, D. & Reid, H. W. (1975). Experimental infection of red grouse with louping-ill virus (flavivirus group) II. Neuropathology. Journal of Comparative Pathology 85, 231-235.
Clutton-Brock, T. H., Coulson, T. & Milner, J. M. (2004). Red deer stocks in the Highlands of Scotland. Nature 429, 261-262.
Duncan, J. S., Reid, H. W., Moss, R., Phillips, J. D. B. & Watson, A. (1978). Ticks, louping-ill and red grouse on moors in Speyside, Scotland. Journal of Wildlife Management 42, 500-505.
Elston, D. A., Moss, R., Boulinier, T., arrowsmith, C. & Lambin, X. (2001). Analysis of aggregation, a worked example: numbers of chicks on red grouse chicks. Parasitology 122, 563-569.
Gilbert, L., Jones, L. D., Hudson, P. J., Gould, E. A., & Reid, H. W. (2000) Role of small mammals in the persistence of Louping-ill virus: field survey and tick co-feeding studies. Medical and Veterinary Entomology, 14, 277-282.
Gilbert, L., Jones, L. D., Laurenson, M. K., Gould, E. A., Reid, H. W., & Hudson, P. J. (2004) Ticks need not bite their red grouse hosts to infect them with louping ill virus. Proceedings of the Royal Society of London Series B-Biological Sciences, 271, S202-S205.
Gilbert, L., Norman, R., Laurenson, K. M., Reid, H. W., & Hudson, P. J. (2001) Disease persistence and apparent competition in a three-host community: an empirical and analytical study of large-scale, wild populations. Journal of Animal Ecology, 70, 1053-1061.
Gray, J. S., Kahl, O., Janetzki, C. & Stein, J. (1992). Studies on the ecology of Lyme disease in a deer forest in County Galway. Ireland Journal of Medical Entomology 29, 915-920.
Hudson, P. J. (1992). Grouse in space and time. Fordingbridge, UK. Game Conservancy.
Hudson, P. J. (1986) The red grouse: the biology and management of a wild gamebird. The Game Conservancy Trust, Fordingbridge.
Hudson, P. J., Dobson, A. P. & Newborn, D. (1998). Prevention of population cycles by parasite removal. Science 282, 2256-2258.
Hudson, P. J., Gould, E., Laurenson, M. K., Gaunt, M., Reid, H., Jones, L., Norman, R., MacGuire, K. & Newborn, D. (1997). The epidemiology of louping-ill, a tick borne infection of red grouse (Lagopus lagopus scoticus). Parasitologia 39, 319-323.
Hudson, P. J., Norman, R., Laurenson, M. K., Newborn, D., Gaunt, M., Jones, L., Reid, H., Gould, E., Bowers, R., & Dobson, A. (1995) Persistence and transmission of tick-borne viruses: Ixodes ricinus and louping-ill virus in red grouse populations. Parasitology, 111, S49-S58.
Hudson, P. J., Rizzoli, A., Rosa, R., Chemin, C., Jones, L. D., & Gould, E. A. (2001) Tick-borne encephalitis virus in northern Italy: molecular analysis, relationships with density and seasonal dynamics of Ixodes ricinus. Medical and Veterinary Entomology, 15, 304-313.
Jenkins, D., Watson, A. & Miller, G. R. (1963). Population studies of red grouse in north-east Scotland. Journal of Animal Ecology 1, 183-195.
Jenkin, D., Watson, A. & Miller, G. R. (1967). Population fluctuations in red grouse. Journal of Animal Ecology 36, 97-122.
Jones, L. D., Gaunt, M., Hails, R. S., Laurenson, K., Hudson, P. J., Reid, H., Henbest, P., & Gould, E. A. (1997) Transmission of louping ill virus between infected and uninfected ticks co-feeding on mountain hares. Medical and Veterinary Entomology, 11, 172-176.
Kirby, A. D. (2003). Invertebrate interaction with red grouse (Lagopus lagopus scoticus). PhD thesis, University of Stirling.
Kirby, A. D., Smith, A. A., Benton, T. G., & Hudson, P. J. (2004) Rising burden of immature sheep ticks (Ixodes ricinus) on red grouse (Lagopus lagopus scoticus) chicks in the Scottish uplands. Medical and Veterinary Entomology, 18, 67-70.
Krebs, J. R. & May, R. M. (1990). Conservation Biology—The Moorland Owners Grouse Nature 343, 310-311.
Kurtenbach, K., Kampen, H, Didij, A., Arndt, S., Seitz, H. M. & Schnaible, U. E. (1995). Infestation of rodents with larval
Ixodes ricinus (Acari: Ixodidae) is an important factor in the
Laurenson, M. K., Hudson, P. J., McGuire, K., Thirgood, S. J., & Reid, H. W. (1997) Efficacy of acaricidal tags and pour-on as prophylaxis against ticks and louping-ill in red grouse. Medical and Veterinary Entomology, 11, 389-393.
Laurenson, M. K., Norman, R., Reid, H. W., Pow, I., Newborn, D., & Hudson, P. J. (2000) The role of lambs in louping-ill virus amplification. Parasitology, 120, 97-104.
Laurenson, M. K., Norman, R. A., Gilbert, L., Reid, H. W., & Hudson, P. J. (2003) Identifying disease reservoirs in complex systems: mountain hares as reservoirs of ticks and louping-ill virus, pathogens of red grouse. Journal of Animal Ecology, 72, 177-185.
Moss, R., & Watson, A. (1985). Adaptive value of spacing behaviour in population cycles of red grouse and other animals. In: Behavioural Ecology, 275-294, Sibley, R. M. & Smith, R. H. (Eds). London, Blackwell.
Moss, R.,& Watson, A. (2001). Population cycles in birds of the grouse family (Tetraonidae) Advances In Ecological Research 32, 53-111.
Norman, R., Bowers, R. G., Begon, M., & Hudson, P. J. (1999) Persistence of tick-horne virus in the presence of multiple host species: Tick reservoirs and parasite mediated competition. Journal of Theoretical Biology, 200, 111-118.
Norman, R., Ross, D., Laurenson, M. K., & Hudson, P. J. (2004) The role of non-viraemic transmission on the persistence and dynamics of a tick borne virus—Louping ill in red grouse (Lagopus lagopus scoticus) and mountain hares (Lepus timidus). Journal of Mathematical Biology, 48, 119-134.
Park, K. J., Robertson, P. A., Campbell, S. T., Foster, R., Russell, Z. M., Newborn, D & Hudson, P. J. “001), The role of invertebrates in the diet, growth and survival of red grouse (Lagopus lagopus scoticus) chicks. Journal of Zoology, London 254, 137-145.
Reid, H. W. (1975). Experimental infection of red grouse with louping-ill virus I. The viraemia and antibody response. Journal of Comparative Pathology 85, 223-229.
Reid, H. W., Duncan, J. S., Phillips, J. D. B., Moss, R. & Watson, A. (1978). Studies on louping-ill virus (flavivirus group) in wild red grouse (Lagopus lagopus scoticus). Journal of Hygiene 81, 321-329.
Redpath, S. M. (1991). The Impact Of Hen Harriers On Red Grouse Breeding Success. Journal Of Applied Ecology 28, 659-671.
SENS (2001). Key Scottish Environmnetal Statistics: 2002 (ed. By J. Landrock and H Snowling). Scottish Executive National Statistics Publications, Scottish Executive Development Department, Edinburgh.
Shaw, D. J., Haydon, D. T., Cattadori, I. M., et al. (2004). The shape of red grouse cycles Journal of Animal Ecology 73, 767-776.
Smith, A. A., Redpath, S. M., Campbell, S. T., & Thirgood, S. J. (2001). Meadow pipits, red grouse and the habitat characteristics of managed grouse moors. Journal of Applied Ecology 38, 390400.
Sonenshine D. E. (1991). Biology of Ticks, vol. 1. Oxford, Oxford University Press.
Sonenshine D. E. (1993). Biology of Ticks, vol. 2. Oxford, Oxford University Press.
Sonenshine D. E. & Mather, T. N. (1994). Ecological dynamics of tick borne zoonoses. Oxford University Press, New York
Thirgood, S. J., Redpath, S. M, Haydon, D. T., et al. (2000). Habitat loss and raptor predation: disentangling long- and short-term causes of red grouse declines. Proceedings of the Royal Society of London Series B-Biological Sciences 267, 651-656.
Watson, A. & Moss, R. (1979). Population cycles in the Tetraonidae. Ornis. Fenn 56, 87-109.
Watson, A., Moss, R., Parr, R., Mountford, M. D. & Rothery, P. (1994). Kin land ownership, differential aggression between kin and non-kin and population fluctuations in red grouse. Journal of Animal Ecology 63, 39-50.
Wilson, M. L., Telford, S. R. III, Piesman, J. & Spielman, A. (1988). Reduced abundance of immature Ixodes dammini (Acari: Ixodidae) following elimination of deer. Journal of Medical Entomology 25, 224-228.

We claim:

1. A method for controlling arthropod pests on fowl comprising affixing to said fowl a pesticide containing element worn by said animal wherein said element is selected from the group consisting of a leg band and wing tag, which said leg band and wing tag comprises a pesticide in an amount effective against an arthropod pest of said fowl.

2. The method of claim 1 wherein said fowl is selected from a mother hen or an adult female nesting bird.

3. The method of claim 2 wherein said fowl comprises a game bird or a pet bird.

4. The method of claim 2 wherein said fowl is selected from the group consisting of quail, pheasants, grouse, canaries, parakeets, macaws, and parrots.

5. The method of claim 4 wherein said fowl is a red grouse.

6. The method of claim 2 wherein said element comprises said leg band.

7. The method of claim 1 wherein said fowl comprises a chick.

8. The method of claim 1 wherein said leg band and wing tag is coated with or impregnated with said pesticide.

9. The method of claim 1 wherein said le band or wing tag comprises metal, natural polymer, synthetic polymer, natural fabric, synthetic fabric, cotton, felt, wool, leather, or combinations thereof.

10. The method of claim 1 wherein said pesticide is selected from the group consisting of an organophosphate, pyrethroid, avermectin, formamidine, and combinations thereof.

11. The method of claim 1 wherein said arthropod pest is selected from the group consisting of acarine and insect ectoparasites of fowl.

12. The method of claim 11 wherein said arthropod pest is a tick.
